(19) United States
(12) Patent Application Publication
(10) Pub. No.: US 2005/0017179 A1
(43) Pub. Date: Jan. 27, 2005

(54) Novel optical method for diagnosis and staging of premalignant and malignant human colonic tissues

(75) Inventors: Shaul Mordechai, Omer (IL); Jed Goldstein, Omer (IL); Shmuel Argov, Omer (IL); Hugo Guterman, Beer-Sheva (IL); Jagannathan Ramesh, Beer-Sheva (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(73) Assignees: BEN-GURION UNIVERSITY OF THE NEGEV, BEER-SHEVA (IL); MOR RESEARCH APPLICATIONS LTD., PETACH TIKVAH (IL)

(21) Appl. No.: 10/846,164

(22) Filed: May 14, 2004

Related U.S. Application Data
(63) Continuation of application No. PCT/IL02/00912, filed on Nov. 14, 2002.

(30) Foreign Application Priority Data
Nov. 15, 2001 (IL) 146821

(51) Int. Cl. G01N 21/35

(52) U.S. Cl. 250/339.08; 702/19

(57) ABSTRACT

The invention presents methods of using Fourier transform infrared microspectroscopy (FTIR-MSP) for distinguishing between normal and abnormal human cells and for diagnosis and digital staging of human cells on biopsied samples. The invention further presents computational methods employing neural network (NN) based classifiers for diagnosis and digital staging of adenomatous polyp and malignant tissue types of biopsied samples of human cells.

### RELATED APPLICATION

This application is a continuation of International Patent Application No. PCT/IL02/00912, filed Nov. 14, 2002, which is here incorporated by reference in its entirety.

### BACKGROUND OF THE INVENTION

1. Field of the Invention

The present invention is directed to the field of cancer treatment. More specifically the invention is directed towards providing a novel method based on Fourier transform infrared microspectroscopy for diagnosis and staging of premalignant and malignant human tissue, in particular colonic tissue.

2. Prior Art

Publications and other reference materials referred to herein, including reference cited therein, are incorporated herein by reference in their entirety and are numerically referenced in the following text and respectively grouped in the appended Bibliography, which immediately precedes the claims.

Colorectal cancer is the second most prevalent cancer type and one of the major causes of morbidity and mortality. It is estimated that 148,300 cases will be diagnosed and 56,600 deaths will occur from colorectal cancer in 2002 in the US [1]. Worldwide, the number of new cases of colorectal cancer has been increasing since 1975 (it was 500,000 in 1975 and increased to 783,000 in 1990) [2]. Men are at higher risk than women by a factor of 3-4 and the peak age of incidence of the disease is at around 60 years. If the colorectal cancer is detected at an early stage, then the five-year relative survival rate is 90%; however only 37% of colorectal cancers are diagnosed at the early stages.

Colorectal cancers have a wide range of neoplasms from benign growths to metastatic states. Polypoid lesion is defined as any mass projecting above the surface of normal mucosa [3]. There are three important types of lesions known: non-neoplastic polyps (hyperplastic), neoplastic polyps (adenomatous polyps) and cancers. The non-neoplastic polyps are not generally considered as precursors of cancer. The adenomatous type has a great deal of clinical significance having a high probability of becoming malignant. The adenomatous polyps are classified into three major subtypes: tubular, tubulo-villous, and villous classified based on epithelial architecture.

Accurate staging of any cancer type is useful for early treatment of premalignant growths and helps the physician in the follow-up schedule. Using current diagnostics methods, in the initial stages, the physician diagnoses the colorectal cancer using endoscopic techniques such as sigmoidoscopy and colonoscopy [4,5]. These techniques are useful in the identification of adenomatous polyps (which are premalignant) as well as malignant areas in the large intestine. The physician accuracy in the diagnosis of non-neoplastic from neoplastic polyp by the magnifying colonoscopy is 88.4% [6] but generally the physician is unable to determine the exact staging of the premalignant or malignant growths. Another routine procedure conducted in the standard diagnosis process is the use of fine needle biopsy followed by standard pathological methods [7]. Even though pathological methods offer information on identification of adenomatous polyp and malignancy, they are highly subjective and inaccurate regarding the staging of malignancy. Since the exact staging of malignancy would be of great assistance to the physician in treating the patient effectively, a method for the digital staging of premalignant and malignant human colonic tissues could be expected to revolutionize the diagnosis of colon cancer thereby improving treatment conditions and eventually leading to higher survival rate for colon cancer patients.

Among the optical methods available in many fields of research, the method of Fourier transform infrared spectroscopy FTIR has become very useful in the field of medicine. Data collection on cells using the conventional FTIR spectrometers however leads to inaccuracy and loss of sensitivity. This problem has been overcome by the innovation of combining light microscopy with FTIR spectrometers, the technique being popularly known as Microscopic FTIR, IR Scope, or FTIR microspectroscopy (FTIR-MSP). With this advancement in instrumentation, application of FTIR in medicine has become a reality.

The basic principle of what is known as “Optical Biopsy” is quite easily understood. Various biomolecular components of the cell have a characteristic IR spectrum, which is rich in structural and functional aspects [8, 9]. The biomolecular fingerprint of cells [10] present in the tissues is altered in the diseased state and these alterations can be detected using IR spectroscopy.

Recently there have been many developments in application of FTIR to the problems of cancer diagnosis [11-13 and U.S. Pat. No. 6,214,550]. An FTIR study of human breast, normal and carcinoma tissues has been carried out and the method of analysis resulted in nearly 100% diagnostic accuracy of carcinoma tissues from normal ones [14]. Staging of breast tumors has also been achieved successfully by FTIR [15]. It has previously been reported that the normal and malignant types of cells can be clearly distinguished using standard formalin fixed tissues of colon cancer patients and FTIR microspectroscopy (FTIR-MSP) [16, 17]. The problem that has not yet been addressed is how to digitally grade pre-malignant and malignant lesions in order to provide more accurate and objective diagnoses.

It is therefore a purpose of this invention to provide a method for providing an objective approach for distinguishing between premalignant and malignant human tissue, particularly colonic tissue.

It is another purpose of this invention to provide a method for the digital staging of premalignant and malignant human tissue, particularly human colonic tissue.

It is a further purpose of this invention to provide a method for the digital staging of premalignant and malignant human tissue, particularly colonic tissue that is objective and more accurate than the existing methods.

It is yet a further purpose of this invention to provide a method that can be used to identify the familial cases from sporadic ones with regard to development of malignancy in any human tissue, particularly colonic tissue.

Further purposes and advantages of this invention will appear as the description proceeds.

### SUMMARY OF THE INVENTION

In a first aspect, the invention is directed towards providing a method of using Fourier transform infrared microspectroscopy (FTIR-MSP) for distinguishing between normal and abnormal human colonic cells on a biopsied sample. The sample can be normal or malignant cells on a tissue sample or benign or adenomatous polyps. The method for distinguishing between normal and abnormal human colonic cells can be carried out manually or carried out automatically using special supporting software and comprises the following steps:
i) obtaining the FTIR-MSP spectrum from a multitude of sites on the sample to obtain a multitude of measurements for the sample;
ii) averaging the measurements to obtain an average spectrum for the sample; and
iii) observing the pattern of the average spectrum in a specified wavenumber region in order to distinguish between normal and abnormal human colonic cells on the biopsied sample. In the examples presented hereinbelow, the specified wavenumber region is between 1000-1200 cm⁻¹.

In another aspect, the invention is directed towards providing a method of using Fourier transform infrared microspectroscopy (FTIR-MSP) for diagnosis and digital staging of human colonic cells on a biopsied sample. The sample can be normal or malignant cells on a tissue sample or benign or adenomatous polyps. The method for diagnosis and digital staging of human colonic cells can be carried out manually or carried out automatically using special supporting software and comprises the following steps:
i) obtaining a multitude of FTIR-MSP spectra from a multitude of sites on the biopsied sample;
ii) adjusting the baseline of each of the spectrum;
iii) normalizing each of the spectrum;
iv) determining the average spectrum from the multitude of spectra;
v) determining the integrated absorbance of a specified wavenumber band of the average spectrum where in the examples presented hereinbelow, the specified wavenumber region is between 1000-1480 cm⁻¹; and
vi) digitally staging the human colonic cells of the biopsied sample according to the value of the integrated absorbance.

In both of the above methods, the sites on the sample are chosen using the following criteria: maximization of the number of epithelial cells and avoidance of regions such as the mucin layer, connective tissue, blood vessels, and lymphoid follicles.

In yet another aspect, the invention is directed towards providing a method for identifying familial cases from sporadic population, comprising the following steps:
i) obtaining a multitude of FTIR-MSP spectra at different sites of the tubular gland (crypt;
ii) obtaining the spectra at the top and at the bottom sites of the normal crypt of the colon cancer patients;
iii) determining the integrated absorbance of a specific wavenumber band of said average spectra; and
iv) identification of the familial cases from sporadic cases from the spectral absorbance differences in a specific wavenumber band.

In a further aspect, the invention is directed towards providing a computational method employing a neural network (NN) based classifier for diagnosis and digital staging of adenomatous polyp and malignant tissue types of biopsied samples of human colonic cells. The computational method is comprised of the following steps:
i) obtaining an FTIR-MSP spectrum of the sample;
ii) applying a wavelet transform to the spectrum;
iii) determining a multitude of wavelet coefficients;
iv) using a selected set of the wavelength coefficients as inputs to a NN based classifier;
v) randomly selecting training and test sets from the same data sets;
vi) repeating the classification a multitude of times, with the same network parameters but with different sets of randomly selected training vectors; and
vii) averaging the results.

In the examples presented hereinbelow, the wavelet transform is the fast wavelet transform (FWT) and the NN based classifier is either the multilayer perceptron (MLP) or the probabilistic neural networks (PNN).

In all of the above methods, the biopsied tissue samples are prepared for measurement using FTIR microscopy by the following steps:
i) cutting the sample into slices having a thickness of 5 to 10 microns;
ii) layering the slices in a water bath;
iii) transferring the slices onto ZnSe crystals; and
iv) drying the slices in an oven.

Although colonic tissue is used throughout this description as the representative tissue, it should be understood that the invention is not limited to colonic tissue and can be exploited with a variety of other tissues, as will be apparent to the skilled person.

All the above and other characteristics and advantages of the invention will be further understood through the following illustrative and non-limitative description of preferred embodiments thereof, with reference to the appended drawings.

### BRIEF DESCRIPTION OF THE DRAWINGS

FIG. 1 presents the FTIR microspectroscopy spectrum of normal and malignant biopsied colonic tissue samples;

FIG. 2 presents the FTIR microspectroscopy spectrum of benign and adenomatous polyps of biopsied colonic tissue samples;

FIG. 3 presents the integrated absorbance (IA) for different stages of adenomatous poly for a group of patients;

FIG. 4 presents the average integrated absorbance for the group of patients of FIG. 3 as a function of polyp dysplasia provided by pathologist;

FIG. 5 presents the average integrated absorbance for the group of patients having various stages of polyp dysplasia 1=hyperplastic; 2=mild; 3=mild-moderate; 4=moderate; 5=moderate-severe, with different stages of polyp dysplasia from the same patient are connected by the dashed lines;

FIG. 6 presents the integrated absorbance (IA) for different stages of biopsied malignant colonic tissue samples from a group of patients, where 0=normal; 1=B1; 2=B2; 3=C2;

FIG. 7 presents the average integrated absorbance for the group of patients shown in FIG. 6;

FIG. 8A presents the FTIR microspectroscopy spectrum of epithelial cells from patients a, b from bottom (solid lines) and top (dashed lines) of normal crypt from a young and advanced patient;

FIG. 8B presents the phosphate content of the epithelial cells at the bottom and top of normal crypts obtained from four patients;

FIG. 8C presents a schematic representation of normal (left) and adenomatous crypt (right) showing cell migration and its corresponding living status, filled and open circles represent the cells in exponentially growing and apoptotic stages respectively;

FIG. 9 presents a summary of the age distribution of the patients included in this study;

FIG. 10 presents the “three classifiers” strategy for diagnosis and digital staging of adenomatous polyp and malignant tissue types; and

FIG. 11 presents the “four classifiers” strategy for diagnosis and digital staging of adenomatous and malignant tissue types.

### DETAILED DESCRIPTION OF PREFERRED EMBODIMENTS

The invention will now be further explained through the illustrative and non-limitative description of preferred embodiments.

For the examples given below, all the FTIR microscopy measurements were performed using a BRUKER EQUINOX spectrometer “IR scope 11” model. The apparatus is equipped with a very highly sensitive Mercury Cadmium Telluride (MCT) detector and equipped with a dual microscope having both visible and IR modes. The MCT detector was cooled to liquid nitrogen temperature before the measurements. The data collection system has a computer controlled X-Y stage facility for chemical mapping purposes. The aperture values of the instrument can be changed between 20 μm to 200 μm. Before each measurement, calibration was performed using a polystyrene sample provided with the instrument by the manufacturer and proper operating condition of the apparatus was confirmed.

The data used in the examples below is derived from tissue specimens preserved in paraffin blocks. The source of these specimens is the biopsy archive of the Pathology Department of the Soroka Medical Center, Beer Sheva, Israel. Thin sections of sample of five to ten microns thickness are suitable for FT-IR microspectroscopy study. In the examples described below 5-10 micron thick tissue sections were cut with an automatic microtome. Successive tissue sections of five-micron thickness were cut for microscopic observation. The cut tissues were layered in a water bath and then transferred onto a microscope slide (zinc-selenium for FT-IR). The slides were dried in a 37° C. oven for 24 hours and then deparaffinization was carried out by treating with xylol followed by immersion in 100% Isopentan for xylol removal. The tissue samples used for FT-IR microspectroscopy study were in the dry state, while the corresponding slides observed by light microscope were stained by Hematoxilin-Eosin for identification of the tissue structure.

Tissue samples were fixed on ZnSe crystals of dimension 2×2 cm² and having 1 mm thickness. During the measurements, a magnification of ×150 was used. The criterion used for site selection was to maximize the epithelial cells on the tissue and to avoid unnecessary regions such as mucin layer, connective tissues, blood vessels, lymphoid follicles, etc. The aperture size used in the measurements was 100 μm covering 5-10 epithelial cells.

Initially, the background spectrum of the ZnSe was collected. After the measurement site was chosen using visible light, the microscope was changed to the IR mode. IR spectra were measured for normal, polyp (benign and adenomatous) and malignant cells. Microscopic FTIR-measurements were performed in transmission mode. The number of co-added scans was increased to 128 to achieve high signal to noise ratio. A database having about 850 measured spectra of normal, polyp and malignant cells at a resolution of 4 cm⁻¹ was created. The measured spectra cover the wavenumber range 600-4000 cm⁻¹ in the mid-IR region. The average analog to digital conversion (ADC) rate was recorded to monitor the quality of spectra with a very low ADC rate corresponding to saturation of the signals.

The spectrometer subtracts the background spectrum from the sample spectrum automatically. The initial spectral analysis was done using OPUS software. The CO₂ band was removed before the baseline correcion. The baseline correction for the spectra was done using a rubber band method. The amide I normalization was applied to all the spectra. The spectra having S/N ratio of above 700 with a proper baseline shape were used for spectral analysis. Reproducibility of the spectra for the same site was examined and it was found to be satisfactory in all measurements.

The data employed in the examples was extracted from 128 patients. The data sets consist of three groups: the control group, the polyp group, and the cancer group: the control group, polyp group and the cancer group. A total of 850 records were available. Table I is a summary of the data employed. The obtained samples were classified with the help of an expert in the field and confirmed by clinical diagnosis assisted by a pathologist.

Definitions

In this application the following definitions are used:

normal=both normal and/or benign cells; and
abnormal=both adenomatous and/or malignant cells.
   Database

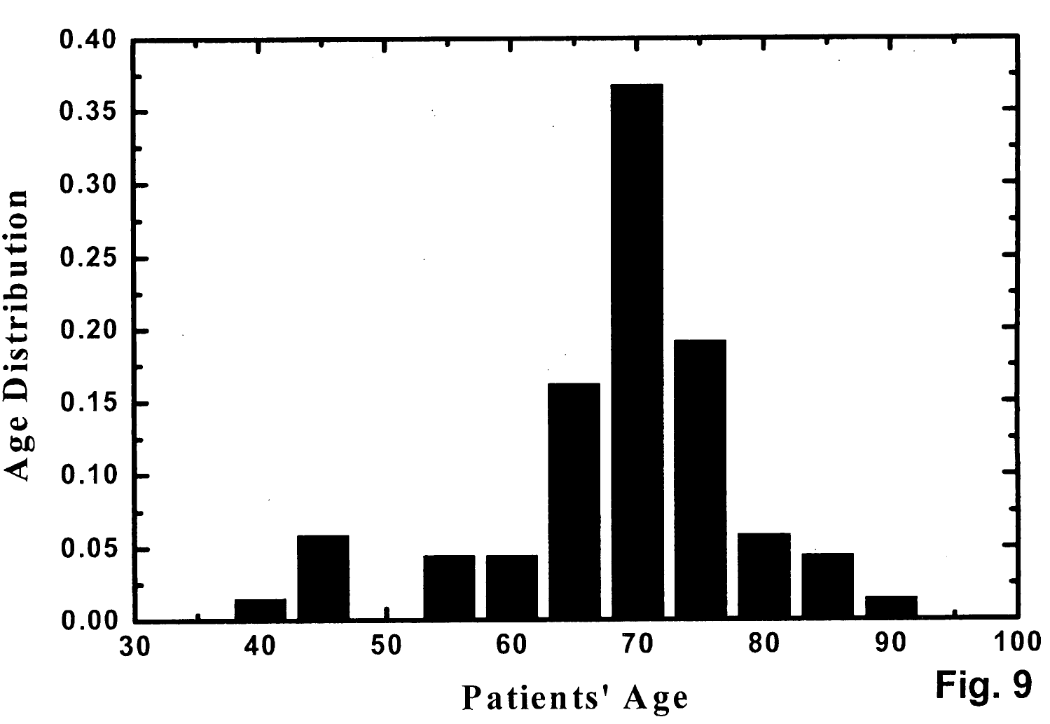

The database consisted of a total of 850 samples from 128 patients (equaly divided between females and males). Several patients contributed with samples in more than just one category. A summary of the data set can be found in Table I and of the age distribution of the patients can be found in FIG. 9.

TABLE I
Data Set

| | Number of | Number of |
|---|---|---|
| Class | samples | patients |
| Normal | 292 | 46 |
| Cancer B1 | 22 | 3 |
| Cancer B2 | 102 | 13 |
| Cancer C2 | 61 | 10 |
| Hyperplastic Polyp | 23 | 4 |
| Mild Polyp | 59 | 9 |
| Mild Moderate Polyp | 30 | 4 |
| Moderate Polyp | 220 | 33 |
| Moderate Severe | 41 | 6 |
| Polyp | | |
| Total | 850 | 128 |

Appropriate feature description of the recorded spectra is considered to be one of the most important components of classification procedures. The result of the feature extraction is a more concise description that still retains most of the characteristics of the spectrum. The use of features rather than the signal itself makes the classification process easier and faster. The present invention accomplishes feature description by employing the computational methods that have been developed for dealing with neural networks.

The term neural network (NN) refers to a network of interconnected neurons [18-20]. An important characteristic of a neural network is its ability to “learn” from examples. Learning (or being “trained”) is achieved through an adaptive process. This ability to learn and build unique structures for a particular problem, without requiring explicit rules or extensive efforts make NN a very attractive computation paradigm. Two neural networks architectures have been employed for colon tissue classification in the present invention: multilayer perceptron (MLP), and probabilistic neural networks (PNN).

The proposed MLP classifier consists of 2-layer feed forward neural network (one hidden layer). The MLP has been commonly used in many NN applications due to its simple implementation [19-22]. The network consists of many processing elements connected in several layers. The output of one processing element is connected to the input paths of other processing elements through connection weights. When presented repetitively with the input and the desired output the MLP organizes internally, gradually adjusting the weights to achieve the desired input/output mapping. Given enough data it is possible to design and teach a MLP with one hidden layer to reproduce the behaviour of any process linear or non-linear [22].

Learning, in the method of this invention, is carried out by a previously described algorithm, which automatically selects the initial conditions of the training [23]. Primarly, the algorithm analyzes the a-priori information contained in the covariance matrix. Based on this information, the algorithm selects and provides an educated guess of the initial weight matrices. Then, an adaptive learning rate backpropagation algorithm is applied [24]. The adaptive learning rate starts with an initial value which is increased and decreased by multipliers to keep the learning rate fast but stable. This algorithm shows better performance than the conventional back propagation in terms of both speed and convergence.

The PNN paradigma have been described by Specht [25]. The network structure is similar to that of the MLP, the main difference being that the sigmoid activation function is replaced by an exponential function. The size of the PNN depends on the number of training samples, as the network adds a hidden unit for each training sample. Therefore, training in the PNN requires only a single pass compared with multiple passes in the back propagation. Probabilistic neural networks are thus orders of magnitude faster to train than the MLP networks.

In the examples below, the performance of the neuron network based classifiers is examined by performing multiscale decomposition of the spectrum. The wavelet transform provides an important tool for signal analysis and feature extraction [26]. It provides a good local representation of the signal in both the time domain and the frequency domain. Unlike the Fourier transform which is global and provides description of the overall regularity of the signals the wavelet transform looks for the spatial distribution of singularities. This transform suits the sampled signal that contains normal singular events such as the peaks observed at the phosphate bands.

In the examples below, the fast wavelet transform (FWT) proposed by Mallat and Zhong [27] is applied. The sampled spectrum is decomposed into an orthogonal set of waveforms that are the dilations, translations and modulations of the Coiflet wavelet (mother wavelet). The Coiflet wavelet was chosen because in practice it showed better results than other common wavelets that were tested. The wavelet transform is computed by convolving the spectrum with these dilated wavelets. The number of scales should be chosen by searching for the optimal signal representation. It was found that scales higher than the first five do not add significant information about the spectrum.

The following examples are provided merely to illustrate the invention and are not intended to limit the scope of the invention in any manner. The results in these examples were obtained by employing 14 wavelet coefficients as features. A Probabilistic Neural Network (PNN) was employed as classifier. Training and test sets were selected randomly from the data set summarized in Table I. Seventy percent of each set was employed for training and the remainder for test. In addition, the simulations were repeated 1000 times, with the same networks parameters but with different sets of randomly selected training vectors, and the results were averaged.

The scientific basis of the present invention is that adenomatous polyp (precancerous) and malignant tissue samples can be considered as abnormal in comparison to controls. The method of differentiating these groups solely depends on the spectral differences in the phosphate (PO₃²⁻) symmetric and asymmetric stretching vibrations. The phosphate containing metabolites such as nucleic acids, phospholipids, and energy precursors are possible contributors to the observed changes in the spectral patterns. Among the cellular molecular machinery, nucleic acids have an important role in the transformation. The literature has abundant evidence for series of genetic changes occuring in the case of colon cancer [30]. These genetic changes are possibly related to the structural and conformational alterations in the DNA which are observed as changes in the spectral patterns between normal and abnormal groups. In addition, the nuclear volume changes have been reported at various steps of malignancy [31]. The examples described hereinbelow are based on a battery of methods of analysis for determining the staging of malignancy and the results obtained using the method of the invention correlate well with the literature.

### EXAMPLE 1

### FTIR-MSP of Normal and Malignant Types from Human Colonic Tissue Samples

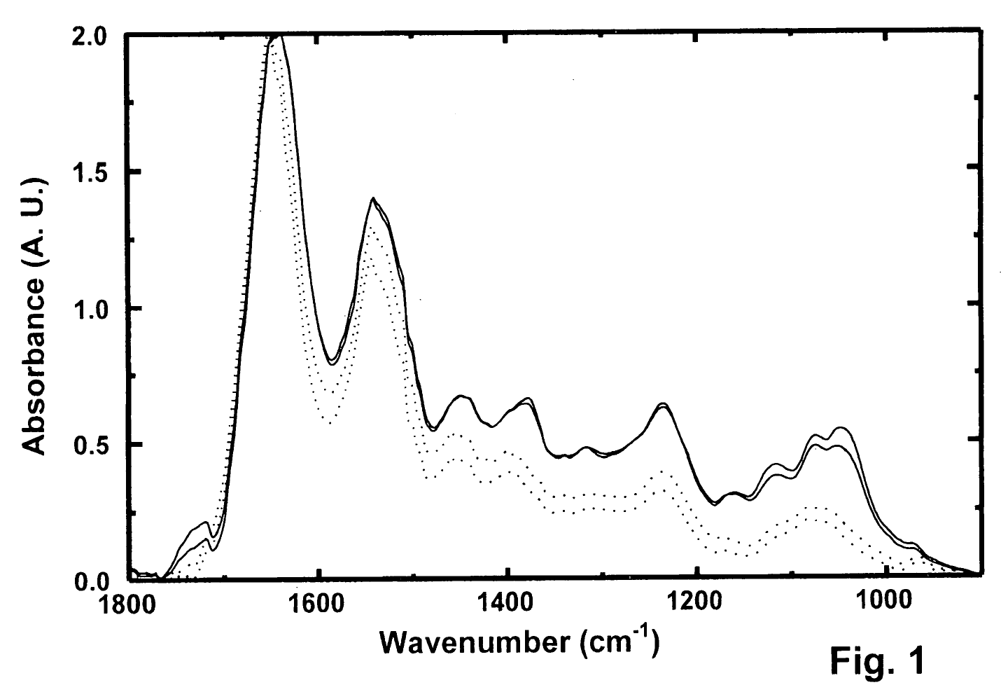

FTIR-MSP spectra of normal (solid lines) and malignant (dotted lines) cell types from biopsy tissue samples of two patients is shown in FIG. 1. Each spectrum in the figure is the average of ten different measurements. The absorbance due to normal tissue is higher than cancerous types in this entire region of the spectrum for these patients. This observation is true in all the other tissue samples obtained from a large number of patients.

There is a distinct change in the pattern in the region between 1000-1200 cm⁻¹ for normal and malignant cells on a given tissue sample. In the spectrum of normal cells, splitting can be clearly observed, which is not present in the case of malignant cells. This behaviour has been observed in most of the cases studied. Therefore it is concluded that this spectral pattern is the signature for normal and malignant cells which can be used to differentiate them. Special supporting software has been created to identify this pattern which will reduce the ambiguities for the pathologist by providing an objective approach to the diagnosis of malignancy on the biopsied tissue samples.

### EXAMPLE 2

### Identification of Benign and Adenomatous Polyps

Polyp growths in the colon can be benign (hyperplastic) or premalignant. Generally, the physicians act with caution and remove polyp growths from the patients because of the ambiguity that results from the present diagnostic tests. Using the method of the invention based on FTIR-MSP analysis of biopsied tissue samples, the benign and adenomatous polyps can be clearly differentiated without any ambiguity.

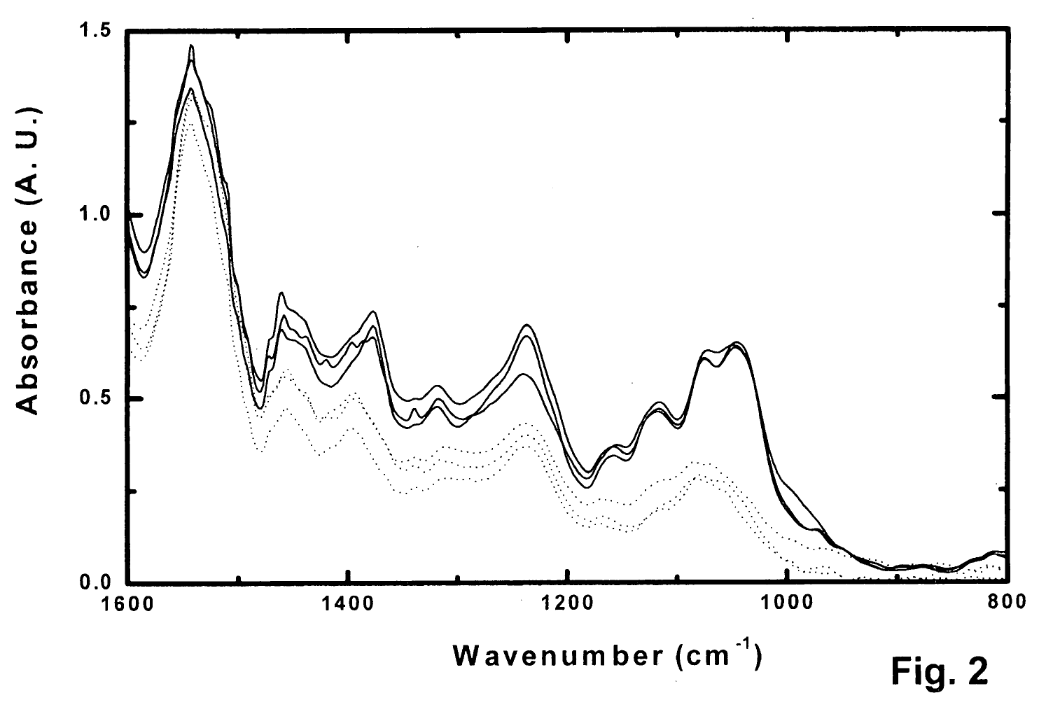

FIG. 2 shows the FTIR-MSP spectra of benign (solid lines) and adenomatous (dotted lines) polyps from three different patients. As in example 1, each spectrum in the figure is the average of ten different measurements. The benign cells behave similarly to normal ones in terms of molecular structure and composition of the cells. The quartet pattern is observed in the phosphate region (1000-1200 cm⁻¹) with benign cells as in the case of normal cells. This pattern completely disappears in the case of an adenomatous polyp. As in example 1, the pattern changes can be identified by suitable software which will reduce the amount of ambiguity in the pathological observations.

### EXAMPLE 3

### Digital Staging of Adenomatous Polyps

According to the literature, 15% of all the premalignant cases have the probability of transforming into malignant in time [28]. Hence it is important to know the exact staging of premalignancy in order to determine how often the patient should undergo follow-up and also which, if any, preliminary treatment should be offered. The present invention provides a method of staging the degree of premalignancy using FTIR-MSP spectra collected on premalignant biopsied tissue samples.

Analysis of the spectra shows that the integrated absorbance between 1000-1480 cm⁻¹ (after normalizing the entire spectrum to the amide I peak) is the best scale for staging of premalignancy in polypoid lesions. The wavelength range between 1000-1480 cm⁻¹ accounts for the various vital cellular metabolites such as nucleic acids, carbohydrates, phospholipids.

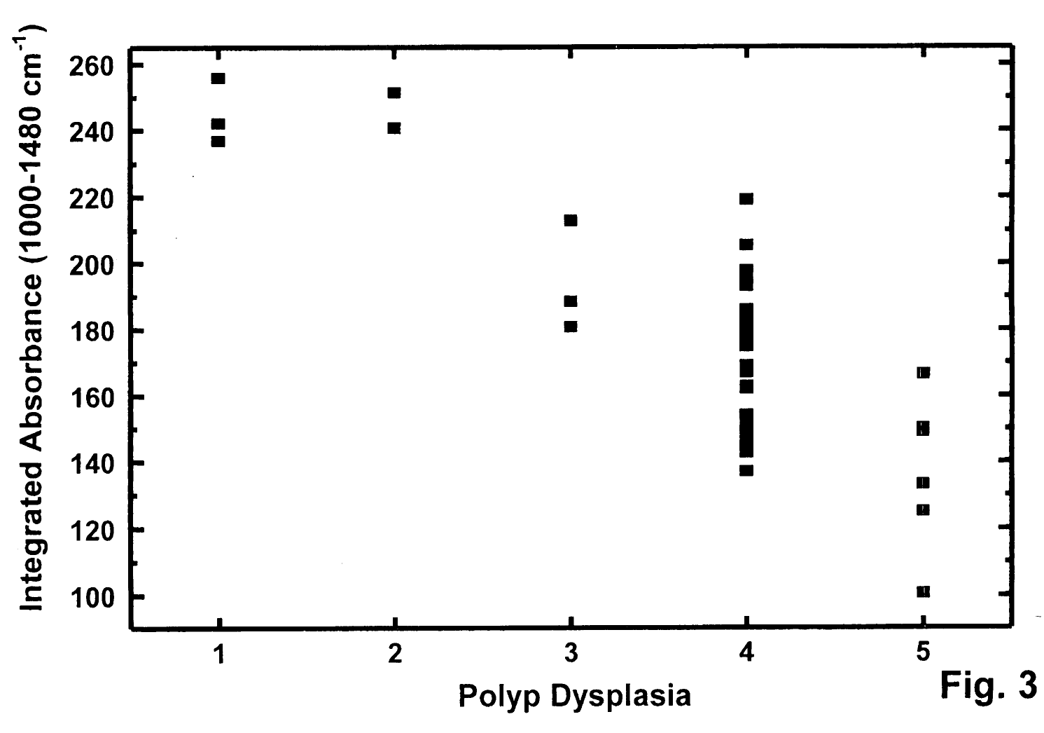

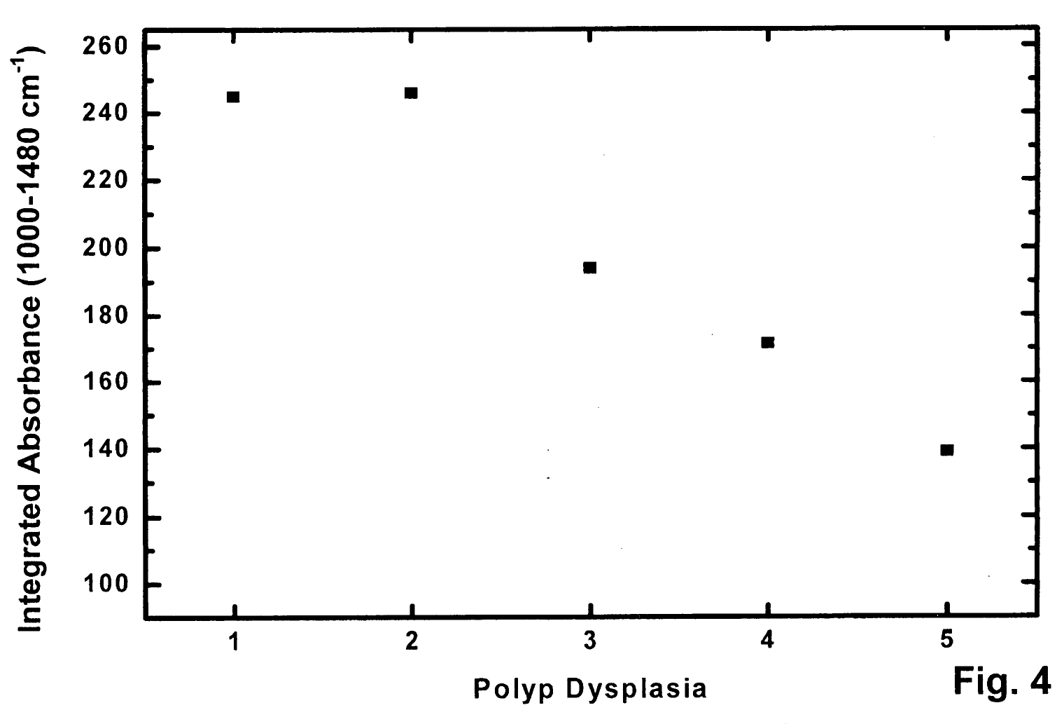

The integrated absorbance between 1000-1480 cm⁻¹ is plotted as a function of polyp displasia as determined by pathologist for a group of patients in FIG. 3 and for the average of all the patients in the group in each category in FIG. 4. The staging into catagories is done by the Astler-Coller method. Category 1 represents the hyperplastic (benign) polyp; 2, mild; 3, mild-moderate; 4, moderate; and 5, moderate-severe. Each square in FIG. 3 represents one patient. There are 33 patients in group 4.

The integrated absorbance between 1000-1480 cm⁻¹ is measured after baseline correction (rubber band method) and normalization by min-max method. The average spectrum of several measurements were used to determine the integrated absorbance. Salient featutes of FIGS. 3 and 4 are:

Even though staging of hyperplastic polyp (1) and the mild cases (2) are the same, they can be differentiated by the specific spectral pattern as discussed in example 2, hereinabove.

Careful scrutiny of mild-moderate (3) and moderate (4) categories indicates that some of the moderate cases actually belong to the mild-moderate class. This shows the drawbacks of current pathological method of staging of polyps and also the power of the optical method.

One of the patients who is graded moderate-severe (5) by the pathologist really belongs to the moderate category (4).

From the last two observations, it can be seen how the irregularities of the pathological method can be corrected by the method of the invention and how, by use of this method, the physician can know the exact status of premalignancy.

The method of staging offers the possibility of making the diagnosis objective and digitized which will give early warning for the patients having an adenomatous polyp in the advanced stages. A scale framed by FTIR-MSP, matching with existing pathological staging is given below (the numbers refer to integrated spectral absorbance after normalization as described above):
a) Mild: 260-230
b) Mild-Moderate: 230-180
c) Moderate: 180-140
d) Mod-Severe: below 140

The borderline cases are to be classified in the more severe category and the digital staging of premalignancy of biopsied tissue samples is presented in the form of scale similar to the blood analysis report. In a given tissue, the most advanced stage of pre-malignant type will be reported.

### EXAMPLE 4

### Digital Staging of Various Stages of Adenomatous Polyps Samples from Individual Patients

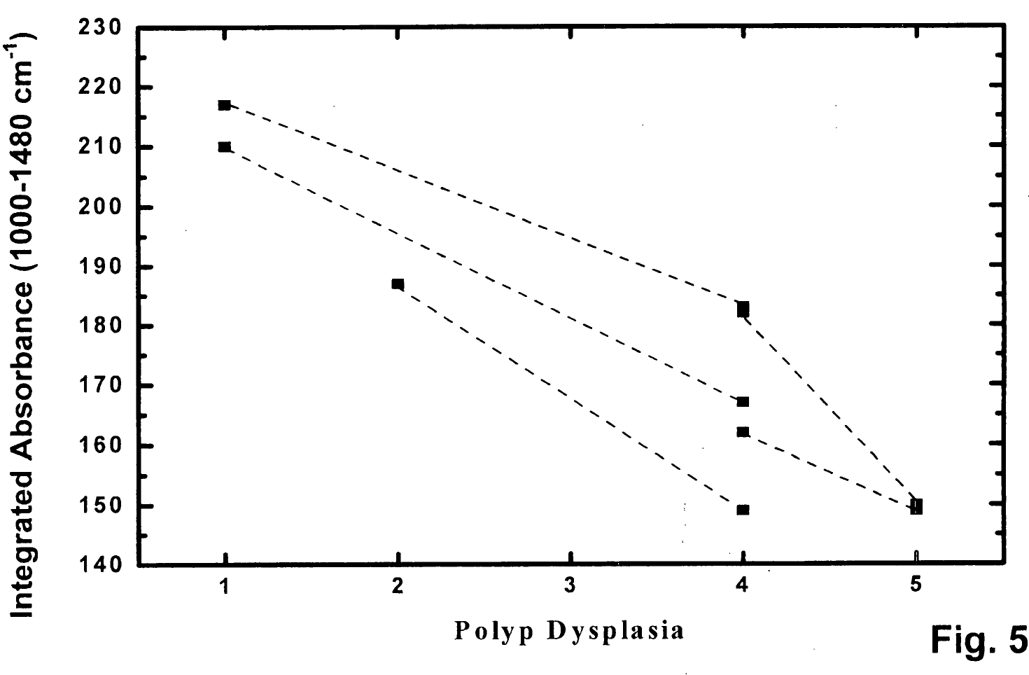

The power of this technique is illustrated by staging adenomatous polyp at different stages existing from each and individual patient. Generally, the pathologist provies the highest staging identified on a given biopsied tissue sample. The method of the invention can differentiate the various stages of polyp in a given biopsed colonic tissue exhibiting the very high sensitivity. Five patients having polyp at various stages are shown in FIG. 5.

1. In the category 1 (hyperplastic polyp), first patient from top had hyperplastic and moderate dysplasia. Our method could differentiate hyperplastic and moderate by spectral pattern changes as well as the difference in integrated absorbance.

2. In the category 1, second patient from top had hyperplastic and moderate dysplasia. Even the closely spaced stages of adenomatous polyp could be identified and staged accurately by the method of the invention.

3. In the same way, the mild to moderate stages could be staged without any ambiguity. One patient having mild & moderate stages; connecting dashed line between categories 2 and 4.

4. In addition, the polyp stages closer to fully malignant ones (two patients having both moderate and moderate-severe; two dashed lines connecting between categories 4 and 5) could be easily staged and it will help the physician to plan more rigorous treatment to avoid the patient from going to fully blown malignant category.

### EXAMPLE 5

### Diqital Staqinq of Malignant Tissue Samples

Knowledge of the stage of malignancy is important for the physician in deciding the course of treatment. The present invention provides an objective and accurate method of staging the degree of malignancy using FTIR-MSP spectra collected on malignant cells of biopsied tissue samples.

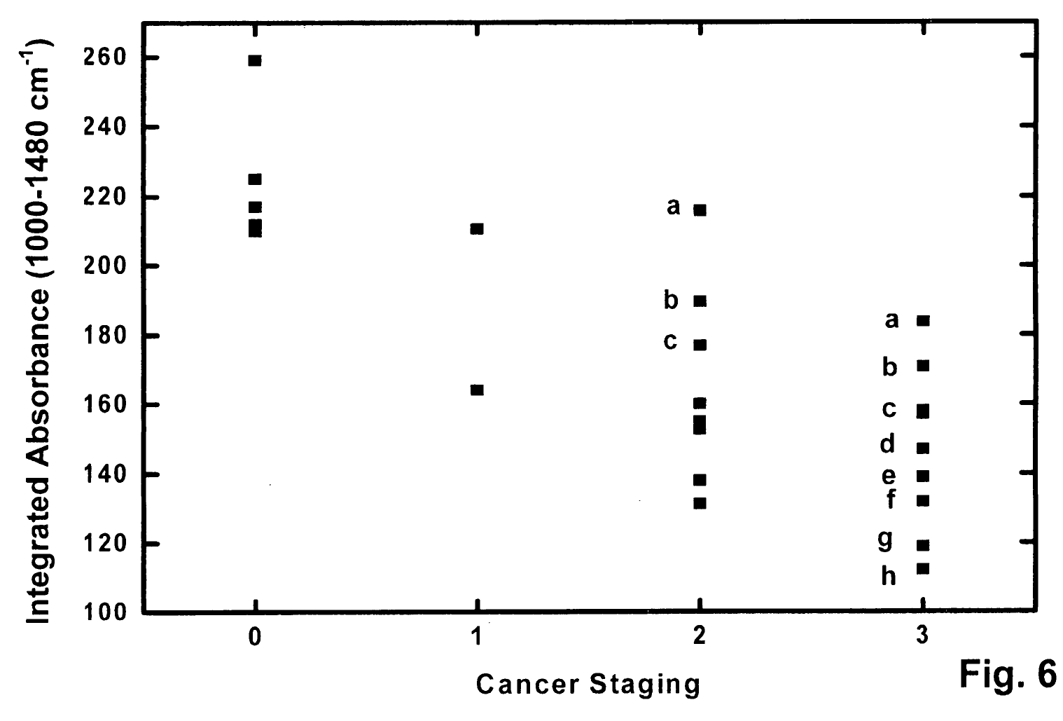

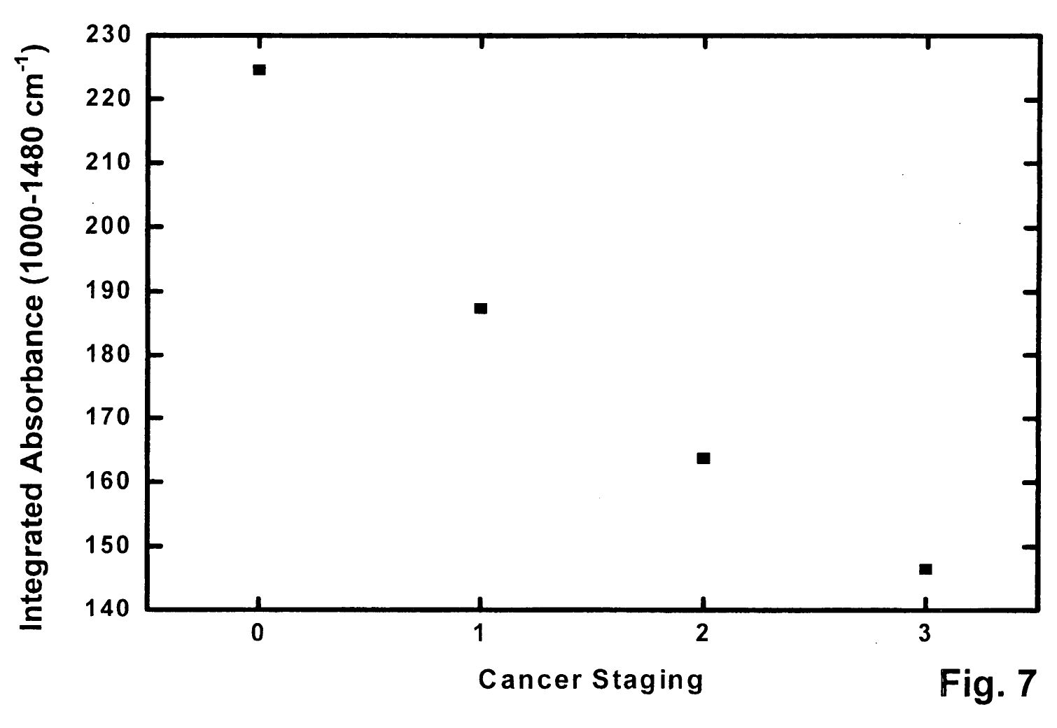

To determine the staging of malignancy, FTIR-MSP spectra of biopsied tissue samples from a group of patients were collected. Integrated absorbance between 1000-1480 cm⁻¹ for different stages of malignancy were then determined. The integrated absorbance between 1000-1480 cm⁻¹ is plotted as a function of malignancy as determined by pathologist for the group of patients in FIG. 6 and for the average of all the patients of the group in each category in FIG. 7. The horizontal axis in FIGS. 6 and 7 represents a scale from 0 to 3 that is used in the method of the invention to specify the stage of malignancy. The staging into catagories was done by the patholoigist using the Astler-Coller staging system. The relationship between the two systems is as follows:

| Present System | Astler-Coller |
|---|---|
| 0 | Control |
| 1 | B1 |
| 2 | B2 |
| 3 | C2 |

The integrated absorbance is measured after baseline correction (rubber band method) and amide I normalization. The average spectrum of several measurements were used to determine the integrated absorbance. Each square in FIG. 6 represents one patient. The averaged integrated absorbance for all patients in each stage of malignancy is presented in FIG. 7. It is clear from the figure that the integrated absorbance decreases significantly with increase in severity of the malignancy. The important observations and conclusions that can be drawn from FIGS. 6 and 7 are:
1. Only two B1 cases are presented because the number of B1 cases is limited my late detection of the malignancy in the patients (only 37% of colon cancer patients are detected in early stages.
2. Patients a, b at stage B2 belong to B1 according to the method of the invention.
3. Patients a, b and c at stage C2 class should be at B2 in contrast to the decisions by the pathologist.
4. Even though the majority of the patients fall in the moderate category (B2), the method of the invention allows arrangement of the patients in different subgrades within this class. In this way, patients can be treated differentially within the same class.
5. The scales based on the integrated absorbance values obtained from FTIR-MSP spectra for the different stages of malignancy in units of integrated spectral absorbance are:
   a) B1: 220-180
   b) B2: 180-150
   c) C2: below 150
6. In a group of measurements (for example 15-20) from different sites of a given tissue, it is reasonable to expect that the most advanced stage of malignant type will be reported by the method.
7. Presentation of staging of malignancy to the physician will be similar to the polyp mentioned in the earlier section (4.3).

### EXAMPLE 6

### Identification of familial cases using FTIR-Microspectroscopy

Identification of familial cases from sporadic ones is vital for planning therapy and continuous monitoring of progression of the disease. In addition, this information will be useful with regard to prevention of colorectal cancer for other family members of the patient. FTIR-MSP spectra were obtained for eleven crypts from four different patients.

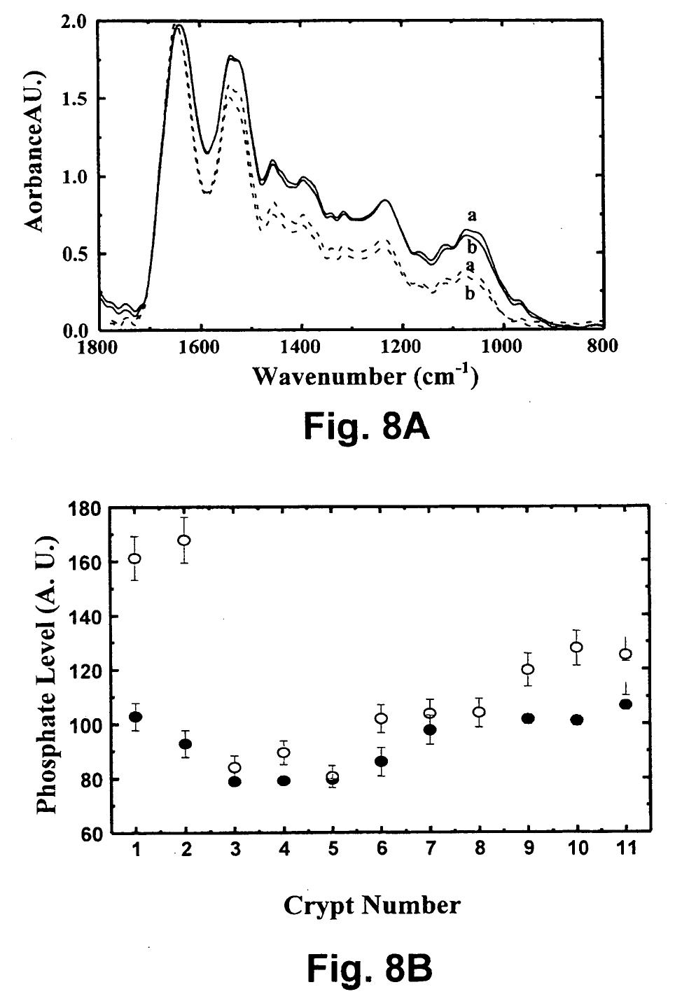

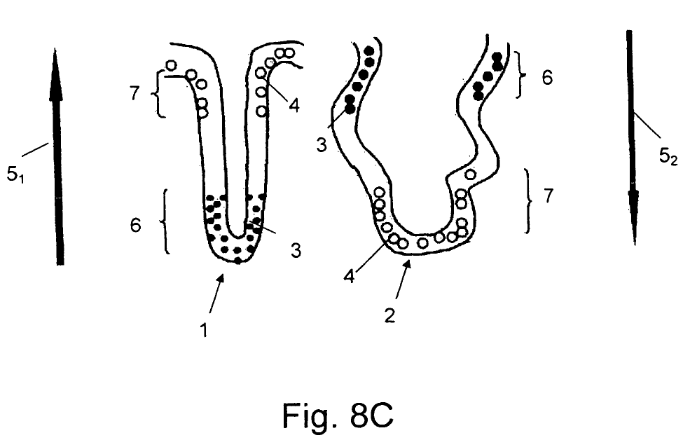

In FIG. 8A are shown the FTIR-MSP spectra of normal crypts from two of these patients (identified by letters a and b in the figure) of different ages. The absorbance for the cells at the bottom site (thick lines) was higher than the top (dotted lines) of the crypt. The absorbance differences between the two crypts for both bottom and top sites were small and hence there was perfect agreement between patients. Notable pattern changes were observed between the bottom and top sites. The cells at bottom showing a doublet structure pattern, between 1000-1100 cm⁻¹, which is absent for the cells at the upper portion of the crypt. This was observed in all of the patients. There was also observed a frequency shift of 4-5 cm⁻¹ in the amide I peak (1650 cm⁻¹) between the two sites for the patient (47 years), whose grading of malignancy was declared by the pathologist to be advanced.

The phosphate content of the epithelial cells at the bottom (open circles) and top (solid circles) of normal crypts obtained from the four patients is presented in FIG. 8B. The phosphate content is derived by integrating the area in the spectrum under the regions corresponding to the symmetric and asymmetric vibration modes of the phosphate group. The results show that the phosphate content of the bottom site of the crypt is higher than at the top in most of the measurements.

The difference in phosphate content between bottom and top was higher for young patients with familial history (Crypt Nos.: 1, 2, and 9 to 11) relative to older patients. Among the younger patients, the difference was the largest for one young patient (Crypt No. 2) having an advanced stage of malignancy. Compared to this patient, the difference was less for the young at the moderate stage of malignancy. However, in older patients (sporadic cases) the difference in phosphate content was not significant irrespective of the grade of the malignancy.

FIG. 8C presents a schematic representation of a normal 1 and an adenomatous crypt 2. In the figure, filled 3 and open 4 circles represent the cells in exponentially growing and apoptotic stages respectively. Also indicated in the figure are the regions of cell proliferation 6, cell death 7, and the direction of cell migration (represented by arrows 5₁ and 5₂ for the respective crypts). Generally, in normal crypts, the epithelial cells are generated in the bottom portion and move further with differentiation and finally exit ending up in apoptosis. A recent model (schematically shown in FIG. 8c) on histogenesis of colonic adenomas presented by Moss et al (32) claims that in adenomas, the proliferation of cells is predominant at lumen (top of the crypt) and apoptosis at the base (bottom of the crypt), a complete reversal of the normal pattern. The concentration of various metabolites being higher in the cells from the bottom site compared to the top of crypt accounts for the absorbance changes between these two sites. This is the reason that the absorbance differences between the bottom and the top of the crypt is reflected in the entire region of the spectrum; and it may be the result of the higher metabolic rate of the rapidly growing cells in the bottom relative to that of the ones undergoing apoptosis at the top of the crypt. This also explains the intensity differences between normal and malignant cells from the bottom of the crypt that have been observed by the inventors (data not shown). The pattern change in the phosphate region is a useful indicator of any deviation from the normal growth status of the cell. This effect has also been observed in the case of malignant cells from the human intestine.

The frequency shifts observed by the inventors for amide I and II between bottom and top sites of the crypts were highest for the young patient who was in an advanced stage of the colon cancer. The magnitude of these frequency shifts was observed to be in the following order: young & advanced>old & advanced>young & moderate>old & moderate. There is a clear trend with age and grade of the malignancy. Probably the conformational associated with some of the proteins would account for these frequency shifts.

### EXAMPLE 7

### RNA/DNA ratio

In order to confirm the validity of the method, detailed analysis on the biological marker derived from the FTIR-MSP spectra was performed. The biological marker was the RNA/DNA ratio of different stages of pre-malignant and malignant types. The RNA/DNA ratio was calculated by using the ratio of bands at wavenumbers 1121/1020. The results indicated that the RNA/DNA ratio increased with the progression of the disease which is in agreement with the literature [29]. In addition, the results on staging correlated well with the histological staging of the expert pathologists.

### EXAMPLE 8

### Computational Methods for Diagnosis and Digital Staging of Adenomatous Polyp and Malignant Tissue Types

The following results were obtained by employing 30 wavelet coefficients as features. The wavelet coefficients were located around the two main peaks of the spectra. In these computations, training and test sets were selected randomly from the same data sets. Seventy percent of each set was employed for training and the remainder for test. In addition, the simulations were repeated 1000 times, with the same networks parameters but with different sets of randomly selected training vectors, and the results were averaged.

a. The performance of the PNN based classifier for normal, cancer and polyp tissue is shown (Table II). It can be seen that the PNN-based classifier shows a success rate of 90.47%, 84.5% and 80.84% for normal, cancer, and polyp tissue, respectively.

TABLE II
FTIR assessment as a confusion matrix: the percentage
of correct and incorrect test diagnoses.

| | Estimated | | |
|---|---|---|---|
| Source | Normal | Cancer | Polyp |
| Normal | 90.47 | 8.62 | 0.91 |
| Cancer | 6.98 | 84.5 | 8.52 |
| Polyp | 0.92 | 18.24 | 80.84 |

b. The performance of the PNN based classifier for normal and abnormal (cancer and polyp) tissue is shown (Table III). The success rate was of 93.61% and 95.38% for normal and abnormal tissue, respectively.

TABLE III
FTIR assessment as a confusion matrix: the
percentage of correct and incorrect test
diagnoses for normal and abnormal tissue.

Estimated

| Source | Normal | Abnormal |
|---|---|---|
| Normal | 93.61 | 6.39 |
| Abnormal | 4.62 | 95.38 |

c. The performance of the classifier for cancer and polyp tissue was 95.14% and 92.73% for cancer and polyp tissue (Table IV), respectively.

TABLE IV
FTIR assessment as a confusion matrix:
the percentage of correct and incorrect test
diagnoses for cancer and polyp tissue.

Estimated

| Source | Cancer | Polyp |
|---|---|---|
| Cancer | 95.14 | 4.86 |
| Polyp | 7.27 | 92.73 |

d. A Probabilistic Neural Network (PNN) was employed to grade cancer and polyp pathologies. As described above, the training and test sets were selected randomly from the same data sets (Table 1).

Table V shows that the PNN based classifier has a success rate of 91.54%, 88.80% and 83.36% for B1, B2 and C2 cancer tissue, respectively.

TABLE V
Cancer tissue (3 classes)

| | B1 | B2 | C2 |
|---|---|---|---|
| B1 | 91.54 | 8.42 | 0.04 |
| B2 | 1.19 | 88.80 | 10.01 |
| C2 | 3.46 | 13.08 | 83.46 |

e. The PNN was also employed to grade mild moderate and moderate polyp tissues (Table VI). The PNN based classifier has a success rate of 82.33%, 82.37%, 89.61% and 83.93% for mild, mild moderate, moderate and moderate severe polyp tissues, respectively.

TABLE VI
Polyp (4 classes)

| | | mild | | |
|---|---|---|---|---|
| | mild | moderate | moderate | moderate |
| | polyp | polyp | polyp | severe |
| mild polyp | 82.33 | 0.76 | 16.10 | 1.57 |
| mild | 2.01 | 82.37 | 12.40 | 3.22 |
| moderate | | | | |
| polyp | | | | |
| moderate | 4.22 | 3.68 | 89.61 | 2.49 |
| polyp | | | | |
| moderate | 0.01 | 2.38 | 13.68 | 83.93 |
| severe | | | | |

The success rate for staging of adenomatous polyp and malignant tissue types will increase with the size of the database.

Possible Classification Schemes

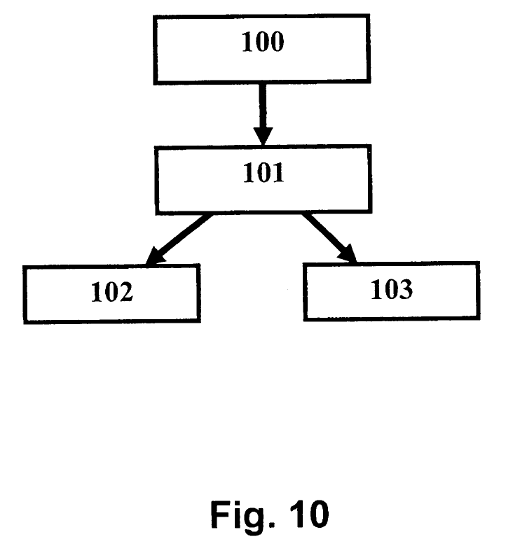

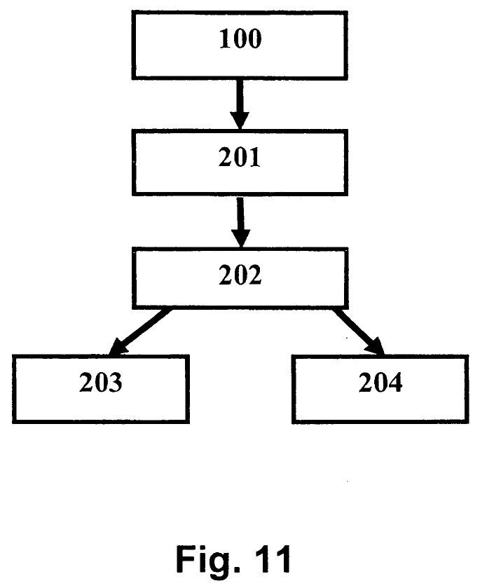

Based on the above results two possible classification strategies are proposed. These two classification strategies are schematically shown in FIG. 10 and FIG. 11.

The first is a “three classifier” strategy in which the results of the FTIR measurements of the samples 100 are, in a first step 101, classified into either “normal” or “cancer and polyp” classifications. In the next step these sets of results are further classified as cancer 102 or polyps 103, according to separate classifications for each group.

The second strategy uses a “four classifier” scheme. In the first step 201, the samples are classified as normal or abnormal, in the second step 202 the abnormal samples are classified as “cancer or polyp”, and in the final step those samples classified as either cancer 203 or polyps 204 are further classified according to separate classifications for each group.

Although embodiments of the invention have been described by way of illustration, it will be understood that the invention may be carried out with many variations, modifications, and adaptations, without departing from its spirit or exceeding the scope of the claims. For example the skilled person can understand how the method described above can be applied to other organs such as breast, prostate, cervix etc.

Bibliography

1. American Cancer Society, Cancer Facts and Figures (2002).
2. Peter, B and Langman, J. S., “ABC of Colorectal Cancer: Epidemiology”, BMJ, 321, (2000), 805-808.
3. Crawford, J. M., “The Gastrointestinal Tract,” Chapter 18 in Pathologic basis of disease, R. S. Cotran, V. Kumar, & T. Collins, Eds. sixth edition, (1999), pp. 827-831 W.B. Saunders Company, PA.
4. Zuber, T. J., “Flexible sigmoidoscopy,” Am. Fam. Physician. 63, (2001), 1375-1380, 1383-1388.
5. Rex, D. K., “Colon tumors and colonoscopy,” Endoscopy. 32, (2000), 874-883.
6. Togashi, k., Konishi, F., Ishizuka, T., Sato, T., Senba, S., and Kanazawa, K “Efficacy of magnifying endoscopy in the differentiatial diagnosis of neoplastic and non-neoplastic polyps of the large bowel”, Dis. Colon. Rectum, 42, [1999], 1602-1608.
7. Adams, J. T., Poulter, C. A. and Pandya, K. J., In: Clinical Oncology, Philip, R (ed) sixth edition, published by American Cancer Society, (1983), 177-189, NY.
8. Mantsch, H. H., and D. Chapman, D., “Infrared Spectroscopy of Biomolecules,” John Wiley, (1996), NY.
9. Jagannathan Ramesh., Salman, A., Hammody, Z., Cohen, B., Gopas, J., Grossman, N., and Mordechai, S., “FTIR microscopic studies on normal and H-Ras oncogene transfected cultured mouse fibroblasts,” Eur. Biophysics. J. (2001), 30 (4), 250-255.
10. Diem, M., Susie Boydston-White, and Chiriboga, L., “Infrared spectroscopy of cells and tissues: Shining light onto a novel subject,” Applied Spectroscopy. 53, (1999), 148-161.
11. Cohenford, M. A., and Rigas, B., “Cytologically normal cells from neoplastic cervical samples display extensive structural abnormalities on IR spectroscopy: implications for tumor biology,” Proc. Natl. Acad. Sci USA. 95, (1998), 15327-15332.
12. Lasch, P., and Naumann, D., “FT-IR microspectroscopic imaging of human carcinoma thin sections based on pattern recognition techniques,” Cell. Mol. Biol (Nosiy-le-grand). 44, (1998), 189-202.
13. Sukuta, S., and Bruch, R., “Factor analysis of cancer Fourier transform infrared evanescent wave fiberoptical (FTIR-FEW) spectrum,” Lasers. Surg. Med. 24, (1999), 382-388.
14. Gao, T., Feng, J., and Ci, Y., “Human breast carcinomal tissues display distinctive FTIR spectra: implication for the histological characterization of carcinomas,” Anal. Cell. Pathol. 18, (1999), 87-93.
15. Jackson, M., Mansfield, J. R., Dolenko, B., Somorjai, R. L., Mantsch, H. H., and Watson, P. H., “Classification of breast tumors by grade and steroid receptor status using pattern recognition analysis of infrared spectra,” Cancer. Detect. Prev. 23, (1999), 245-253.
16. Jagannathan Ramesh., Salman, A., Argov, S., Goldstein, J., Sinelnikov, I., Walfisch, S., Guterman, H., and Mordechai, S., “FTIR Microscopic studies on mormal, polyp and malignant human colonic tissues,” Subsurface Sensing Technologies and Applications, 2, (2001), 99-117.
17. Salman, A., S. Argov, S., Jagannathan Ramesh, Goldstein, J., Igor, S., Guterman, H., Shaul, M., “FTIR microscopic characterization of normal and malignant human colonic tissues”, Cell. Mol. Biol (Nosiy-le-grand), in press (2001).
18. Zupan, J., Gasteiger J., “Neural networks: A new method for solving chemical problems or just a passing phase?”, Anal. Chim. Acta. 248, (1991) 1-30.
19. Lippmann, R. P., “An Introduction to Computing with Neural Nets”, IEEE Proc. ASSP. (1987) 4-22.
20. Wasserman, P. D., “Neural computing: theory and practice”, Van Nostrand Reinhold Press. (1989), NJ.
21. Widrow, B. and Lehr, M. A., “30 years of adaptive neural networks: perceptron, madaline, and backpropagation”, IEEE Proc. 78, (1990), 1415-1442.
22. Cybenko, G., “Approximation by superpositions of a sigmoidal function”, Math. Control Signals Systems, 2, (1989), 303-314.
23. Guterman, H., “Application of Principal Component Analysis to the design of Neural Networks. Neural, Parallel and Scientific Computations”, 2, (1994), 43-54.
24. Volg, T. P., Mangis, J. K., Rigler, A. K., Zink, W. T., and Alkon D. L., “Accelerating the convergence of the backpropagation method”, Biological Cybernetics, 59, (1988) 257-263.
25. Specht, D. F., “Probabilistic neural networks and polynomial Adaline as complementary techniques for classification”, IEEE Trans. Neural Networks, 1 (1990) 111-121.
26. Mallat, S. G., “A wavelet tour to signal processing”, Academic Press, (1998), NY.
27. Mallat, S. G., and Zohng, S. “Characterization of signal multiscale edges”, IEEE Trans. Pattern Anal. Machine Intell. 10, (1992) 710-732.
28. Vamosi-Nagy, I., and Koves, I., “Correlation between colon adenoma and cancer”, Eur. J. Surg. Oncol., 19, (1993), 619-624.
29. Andrus, P. G and Strickland, R. D., “Cancer staging by Fourier transform infrared spectroscopy”, Biospectroscopy 4, (1998), 37-46.
30. Ian, T., Mohammad, I., and Marco, N., “Molecular genetics of colon cancer”, Cancer and Metastasis Reviews, 16, (1997), 67-79.
31. Backman, V., Wallace, M. B., Perelman, L. T., and Arendt., J. T et.al “Detection of preinvasive cancer cells”, Nature, 406, (2000), 35-36.
32. Moss S. F, Liu T. C, Petrotos A, Hsu T. N, Gold L. I, and Holt P. R (1996) “Inward Growth of Colonic Adenomatous Polyps”: Gastroenterology, 111, (1996), 1425-1432.

1. A method of using Fourier transform infrared microspectroscopy (FTIR-MSP) for distinguishing between normal and abnormal human cells on a biopsied sample, wherein said sample is chosen from the group:
normal cells on a tissue sample;
malignant cells on a tissue sample;
benign polyps; and
adenomatous polyps.

2. A method according to claim 1, comprising the following steps:
obtaining the FTIR-MSP spectrum from a multitude of sites on the sample to obtain a multitude of measurements for said sample;
averaging said measurements to obtain an average spectrum for said sample; and
observing the pattern of said average spectrum in a specified wavenumber region in order to distinguish between normal and abnormal human cells on said biopsied sample.

3. A method according to claim 2, wherein observing the pattern of said average spectrum in a specific wavenumber region in order to distinguish between normal and abnormal human cells on said biopsied sample is carried out automatically using special supporting software.

4. A method, according to claim 1, for identifying familial cases from sporadic population, comprising the following steps:
obtaining a multitude of FTIR-MSP spectra at different sites of the tubular gland (crypt);
obtaining the spectra at the top and at the bottom sites of the normal crypt of the colon cancer patients;
determining the integrated absorbance of a specific wavenumber band of said average spectra; and
identification of the familial cases from sporadic cases from the spectral absorbance differences in a specific wavenumber band.

5. A method according to claim 1, wherein the human cells are human colonic cells.

6. A method according to claim 2, wherein the human cells are human colonic cells and the specified wavenumber region is between 1000-1200 cm⁻¹.

7. A method of using Fourier transform infrared microspectroscopy (FTIR-MSP) for diagnosis and digital staging of human cells on a biopsied sample, wherein said sample is chosen from the group:
normal cells on a tissue sample;
malignant cells on a tissue sample;
benign polyps; and
adenomatous polyps.

8. A method according to claim 7, comprising the following steps:
obtaining a multitude of FTIR-MSP spectra from a multitude of sites on the biopsied sample;
adjusting the base line of each of said spectrum;
normalizing each of said spectrum;
determining the average spectrum from the multitude of spectra;
determining the integrated absorbance of a specified wavenumber band of said average spectrum; and
digitally staging the human cells of said biopsied sample according to the value of said integrated absorbance.

9. A method according to claim 8 wherein digitally staging of the human cells of the biopsied sample according to the value of the integrated absorbance is carried out automatically using special supporting software.

10. A method according to claim 2, wherein the sites on the sample are chosen using the following criteria:
maximization of the number of epithelial cells; and
avoidance of regions such as:
mucin layer;
connective tissue;
blood vessels; and
lymphoid follicles.

11. A computational method employing a neural network (NN) based classifier for diagnosis and digital staging of adenomatous polyp and malignant tissue types of biopsied samples of human cells comprised of the following steps:
obtaining an FTIR-MSP spectrum of said sample;
applying a wavelet transform to said spectrum;
determining a multitude of wavelet coefficients;
using a selected set of said wavelength coefficients as inputs to a NN based classifier;
randomly selecting training and test sets from the same data sets;
repeating the classification a multitude of times, with the same network parameters but with different sets of randomly selected training vectors; and
averaging the results.

12. A method according to claim 11, wherein the wavelet transform is the fast wavelet transform (FWT).

13. A method according to claim 11, wherein the NN based classifier is chosen from the group consisting of:
multilayer perceptron (MLP); and
probabilistic neural networks (PNN).

14. A method according to claim 1, wherein the biopsied tissue samples are prepared for measurement using FTIR microscopy by the following steps:
cutting said sample into slices having a thickness of 5 to 10 microns;
layering said slices in a water bath;
transferring said slices onto ZnSe crystals
drying said slices in an oven;

15. A method according to claim 7, wherein the human cells are human colonic cells.

16. A method according to claim 8, wherein the human cells are human colonic cells and the specified wavenumber region is between 1000-1480 cm⁻¹.
